(19) United States
(12) Patent Application Publication
(10) Pub. No.: US 2024/0148947 A1
(43) Pub. Date: May 9, 2024

(54) ULTRASONICALLY POWERED SENSING DEVICE

(71) Applicants: Fresenius Medical Care Holdings, Inc., Waltham, MA (US); Fresenius Medical Care Deutschland GmbH, Bad Homburg (DE)

(72) Inventors: David Yuds, Waltham, MA (US); Martin Crnkovich, Concord, MA (US); Paul Chamney, Tring (GB)

(21) Appl. No.: 17/980,054

(22) Filed: Nov. 3, 2022

(51) Int. Cl. A61M 1/28; H02J 50/15

(52) U.S. Cl. CPC A61M 1/285; H02J 50/15; A61M 2205/0294; A61M 2205/04; A61M 2205/3344

(57) ABSTRACT

Disclosed herein are embodiments of a device that can be powered or charged via ultrasonic energy conducted from another device via a fluid connection. In particular, disclosed herein is a pressure sensor for use with an automatic peritoneal dialysis (APD) cycler wherein power is transferred from the APD cycler to the sensor via ultrasonic wave transmitted through a dialysate fluid. A piezoelectric transducer is used in the device to convert the kinetic energy of the ultrasonic waves in to electrical energy that can be used to power the device or charge a power storage element within the device.

### FIELD OF THE DISCLOSURE

The disclosure generally relates to sensors, and more particularly to wearable or implanted sensors used for medical purposes.

### BACKGROUND

In many applications, remote sensors are used to measure and report environmental conditions. Often, such sensors report readings wirelessly via, for example, a Wi-Fi, Bluetooth or near-field communication (NFC) connection. However, powering such sensors is often an issue. Configuring the devices with a replaceable or rechargeable battery is often not feasible or cost-effective because the location of the sensor makes it difficult or impossible to either replace the battery or to charge the battery via connection to an external power supply.

Dialysis machines are known for use in the treatment of renal disease. The two principal dialysis methods are hemodialysis (HD), in which the patient's blood is passed through a dialyzer of an HD machine, and peritoneal dialysis (PD). During PD, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream. Automated PD machines, called APD cyclers, are designed to control the entire PD process so that it can be performed at home, usually overnight, without clinical staff in attendance.

During the infusion of fresh dialysate and the draining of spent dialysate phases, the APD cycler monitors the patient's peritoneal pressure as affected by the cycler's pumping actions. During the dwell phase, when the patient is no longer connected to the APD cycler, the patient's peritoneal volume increases as the fluids diffuse into it, increasing the peritoneal pressure. By comparing the fill volume and the drain volume, the APD cycler can calculate the ultrafiltration volume after the subsequent drain cycle is complete. This ultrafiltration volume provides a better understanding of the treatment efficacy in terms of the quality of the peritoneum's membrane, the suitability of the dialysate concentration and the time it dwells in the patient.

Because there are so many factors involved in determining the efficacy of the PD treatment, it is difficult to understand exactly how to optimize the patient's prescription for the shortest dwell times, lowest dialysate (glucose) strength, and the fluid transport characteristics of the patient's peritoneum. This is further complicated during the daytime exchanges when the patient is away from the APD cycler because there is no way for the APD cycler to monitor the pressures when disconnected from the patient.

To overcome this challenge, the patient may be provided with a battery powered pressure sensor that remains connected to the patient's catheter. Such a device would contain at a minimum a pressure sensor, a communication module and a power supply, and may be implanted internally in the patient or worn externally. In either case, currently available options for powering the sensor are less than optimal. Powering the sensor with a replaceable battery puts more strain on the catheter because the battery housing adds weight and takes up critical volume. Additionally, the battery itself is not optimized to fit in smallest space possible. Because many patients already have neuropathy issues, manipulating a tiny battery into a small device would be especially problematic. Powering the sensor with a rechargeable battery that must be connected to an external power supply via an electrical wire directly exposes the patient to risk of injury or electrocution and is therefore unacceptable. Lastly, using a disposable device requires a larger, heavier battery for longer device life and is not cost-effective.

As described, powering the pressure sensing device used in this application suffers from the same deficiencies identified with respect to other remote sensors. Therefore, it would be desirable to provide means for powering such devices or for recharging a power storage element in such devices that does not involve changing the power storage element or requiring the power storage element to be connected to an external power supply.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

During PD dialysis, the APD cycler described in the Background section transfers a fluid (i.e., the dialysate) from the cycler to the peritoneum of the patient. In accordance with an exemplary embodiment of the present disclosure, power can be transferred from the APD cycler to the remote sensor via ultrasonic energy conducted from the APD cycler to the remote sensor via the fluid. The APD cycler generates ultrasonic waves which are propagated via the fluid to the remote sensor. A piezoelectric transducer in the sensor transforms the ultrasonic waves into electrical power which can be used to either power the sensor directly or to charge a power storage element on the device for use when the sensor is disconnected from the APD cycler. The piezoelectric transducer uses the piezoelectric effect to convert kinetic or mechanical energy (e.g., the ultrasonic waves), via crystal deformation, into electrical energy.

In a first example, a device comprises a fluid connection to a second device and circuitry for converting kinetic energy of ultrasonic waves transmitted to the device through the fluid connection into electrical energy.

In the first example, or any other example disclosed herein, the device further comprises a power storage element to store the electrical energy.

In the first example, or any other example disclosed herein, the device further comprises one or more sensing elements and a communications module, wherein the one or more sensors and the communications modules are powered by the electrical energy.

In the first example, or any other example disclosed herein, the circuitry of the device comprises a piezoelectric transducer a rectifier and one or more capacitors to store the electrical energy.

In the first example, or any other example disclosed herein, the device further includes wherein the communication module broadcasts readings from the one or more sensors to the second device or to a cloud-based server.

In the first example, or any other example disclosed herein, the device further includes wherein the second device generates the ultrasonic waves and transmits them to the device through the fluid connection.

In the first example, or any other example disclosed herein, the device further includes wherein the device is connected to the second device via tubing carrying the fluid connection.

In the first example, or any other example disclosed herein, the device further includes wherein the second device is a peritoneal dialysis machine and further wherein the fluid connection comprises a dialysate.

In the first example, or any other example disclosed herein, the device further includes wherein the one or more sensors comprises a pressure sensor for sensing pressure of the dialysate within a catheter of a patient.

In the first example, or any other example disclosed herein, the device further includes wherein the device is implanted in the body of the patient.

In a second example, a method for powering a device comprises receiving ultrasonic waves via a fluid connection to a second device and converting kinetic energy of the ultrasonic waves into electrical energy.

In the second example, or any other example disclosed herein, the method further comprises storing the electrical energy in a power storage element.

In the second example, or any other example disclosed herein, the method further includes wherein the device comprises one or more sensing elements and a communications module and the method further comprises powering the one or more sensors and the communications modules using the stored electrical energy.

In the second example, or any other example disclosed herein, the method further includes wherein step of converting the kinetic energy of the ultrasonic waves into electrical energy is performed by circuitry comprising a piezoelectric transducer a rectifier and one or more capacitors to store the electrical energy.

In the second example, or any other example disclosed herein, the method further includes wherein the communication module broadcasts readings from the one or more sensors to the second device or to a cloud-based server.

In the second example, or any other example disclosed herein, the method further includes wherein the second device generates the ultrasonic waves and transmits them to the device through the fluid connection.

In the second example, or any other example disclosed herein, the method further includes wherein the device is connected to the second device via tubing carrying the fluid connection.

In the second example, or any other example disclosed herein, the method further includes wherein the second device is a peritoneal dialysis machine and further wherein the fluid connection comprises a dialysate.

In a second example, or any other example disclosed herein, the method further includes wherein the one or more sensors comprises a pressure sensor and the method further comprises sensing pressure of the dialysate within a catheter of a patient and broadcasting the sensed pressure via the communication module.

In the second example, or any other example disclosed herein, the method further includes wherein the device is implanted in the body of the patient.

### BRIEF DESCRIPTION OF THE DRAWINGS

By way of example, specific embodiments of the disclosed methods and devices will now be described, with reference to the accompanying drawings, in which:

FIG. 1A is a block diagram illustrating an exemplary embodiment of a dialysis system;

FIG. 1B is a block diagram illustrating an example of an embodiment of a dialysis machine and a controller;

FIG. 2 illustrates an example of an embodiment of a dialysis machine (i.e., an APD cycler) that can be used in the dialysis system of FIG. 1A and with exemplary embodiments of the present disclosure; and

FIG. 3 is a schematic diagram of one exemplary embodiment of the present disclosure for use with an APD cycler.

### DETAILED DESCRIPTION

The systems and methods of the present embodiments will now be described more fully with reference to the accompanying drawings, in which several exemplary embodiments are shown. The subject matter of the present disclosure, however, may be embodied in many different forms and types of methods and devices for dialysis machines, other potential medical devices and treatments and with remote sensors used in other, non-medically-related applications, and should not be construed as limited to the embodiments set forth herein. Rather, the disclosed exemplary embodiments are provided so that this disclosure will be thorough and complete, and will fully convey the scope of the subject matter to those skilled in the art. In the drawings, like numbers refer to like elements throughout.

Exemplary embodiments are described herein in the context of a PD dialysis machine. However, as would be realized by one of skill in the art, the disclosed embodiments may be used with any remote sensor, medical or non-medical, that is used in close proximity to the transfer of a fluid being from one point to another. As would be further realized by one of skill in the art, the fluid may be any fluid, for example, dialysate, blood, water, etc.

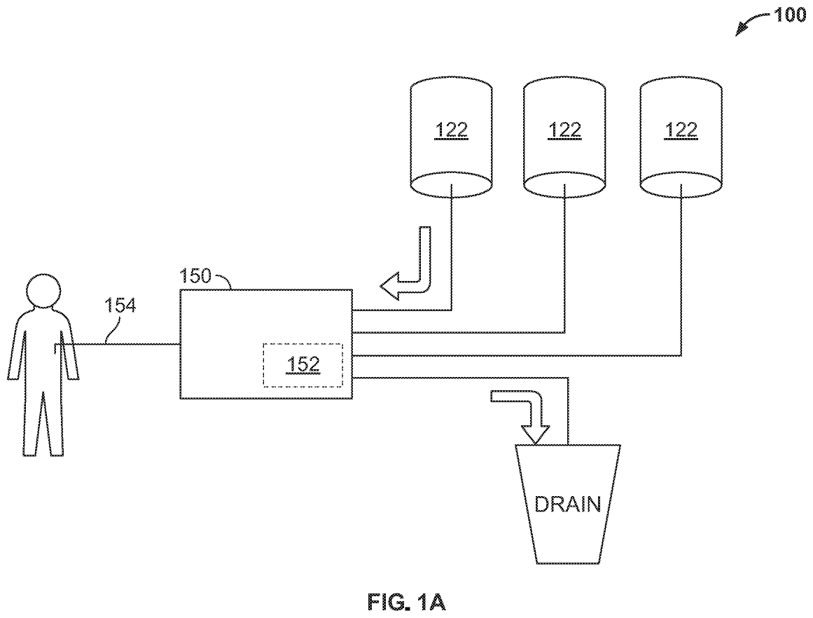

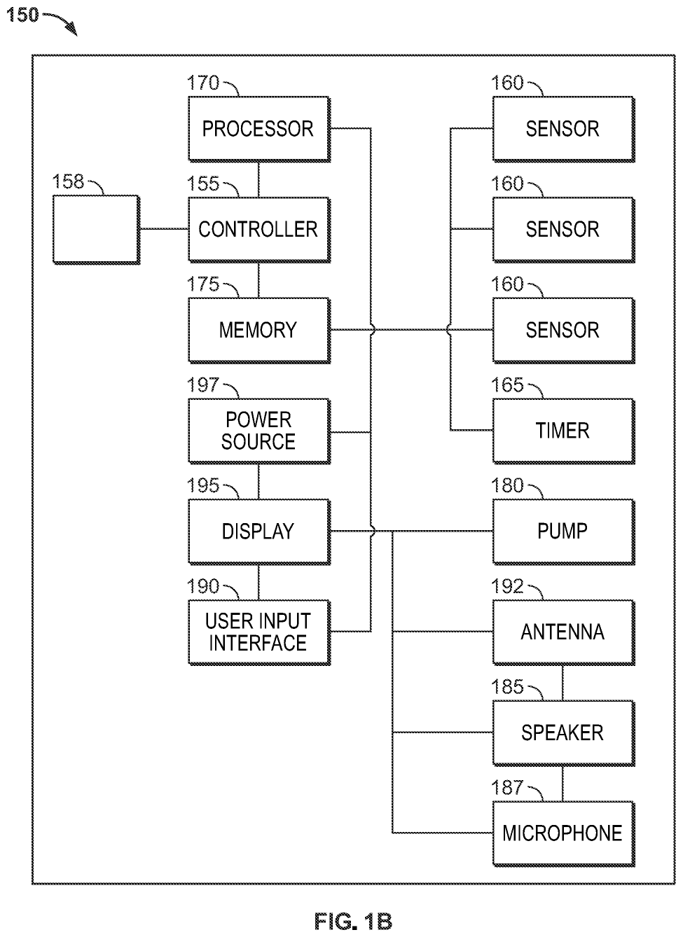

Referring to FIG. 1A, a dialysis system 100 may include a PD dialysis machine 150, for flowing fresh dialysate into a patient and draining used dialysate out of the patient. During treatment, a volume of dialysate may enter the patient's abdomen and remain for a period of time (i.e., the dwell time). During the dwell time, the dialysate may flow across the peritoneum and absorb contaminants and/or particulates from a patient's blood and exchange substances and fluids (e.g., electrolytes, urea, glucose, albumin, osmotically active particles, and other small molecules). At the end of the dwell time, the used dialysate is drained out of the patient's abdomen and purged to a drain connected to the tubing (e.g., the drain line). The exchange of fresh dialysate and used dialysate after a dwell time may occur for several cycles depending on the patient's treatment regimen.

One or more dialysate sources 122 may be connected to the dialysis machine 150. In some embodiments, as illustrated, the dialysate source(s) 122 may be dialysate bags that are hung near PD machine 150 to improve air content management, as any air content is disposed by gravity to a top portion of the dialysate bags 122. Valves may be attached to a bottom portion of the dialysate bags 122 so fluid is drawn out and the delivery of air delivery is minimized.

The patient line 154 may be connected to a patient's abdomen via a catheter and may be used to pass dialysate back and forth between the dialysis machine 150 and the patient's peritoneal cavity during use. The drain line may be connected to a drain or drain receptacle and may be used to pass dialysate from dialysis machine 150 to the drain or drain receptacle during use. Although the system described herein is discussed principally in connection with the use of dialysate bags 122 as the dialysate source, it is noted that, in other embodiments, different dialysate sources may be used. For example, in other embodiments, the dialysate source may include one or more containers in which dialysate is mixed and/or otherwise prepared at the PD cycler from a dialysate concentrate. See, for example, U.S. Pat. No. 10,076,599 (Eyrard et al.), entitled “Dry Peritoneal Dialysis Concentrate System,” which is incorporated by reference herein in its entirety.

Dialysis machine 150 may include components 152 for the generation of ultrasonic waves in accordance with the present disclosure and for coupling the ultrasonic waves to conduit 154, which transfers fluid between the dialysis machine 150 and the patient.

Referring to FIG. 1B, a block diagram of an exemplary embodiment of a dialysis machine such as, for example, dialysis machine 150 and a controller 155 in accordance with the present disclosure are shown. The machine 150 may be a home dialysis machine (e.g., a PD machine), for performing a dialysis treatment on a patient, and may be included in the system 100 described above with respect to FIG. 1A. The controller 155 may automatically control execution of a treatment function during a course of dialysis treatment. The controller 155 may be operatively connected to the sensors 160 and deliver a signal to execute a treatment function (e.g., transferring dialysate from the dialysate bag 122 to the patient), or a course of treatment associated with various treatment systems. In some embodiments, a timer 165 may be included for timing triggering of the sensors 160.

In some embodiments, dialysis machine 150 may also include a processor 170, and memory 175, the controller 155, the processor 170, and/or the memory 175, or combinations thereof of the machine 150, may receive signals from the sensor(s) 160 indicating various parameters. Each fluid bag (e.g., the dialysate bags 122) may contain an approximate amount of dialysate, wherein “approximate amount” may be defined as a 3L fluid bag containing 3000 to 3150 mL, a 5L fluid bag containing 5000 to 5250 mL, and a 6L fluid bag containing 6000 to 6300 mL or any other size of fluid bag. The controller 155 may also detect the connection to all fluid bags 122.

Communication between the controller 155 and the treatment system may be bi-directional, whereby the treatment system acknowledges control signals, and/or may provide state information associated with the treatment system and/or requested operations. For example, system state information may include a state associated with specific operations to be executed by the treatment system (e.g., trigger pump to deliver dialysate, trigger pumps and/or compressors to deliver filtered blood, and the like) and a status associated with specific operations (e.g., ready to execute, executing, completed, successfully completed, queued for execution, waiting for control signal, and the like).

In some embodiments, the dialysis machine 150 may include at least one pump 180 operatively connected to the controller 155. During a treatment operation, the controller 155 may control the pump 180 for pumping fluid (e.g., fresh and spent dialysate), between the dialysis machine 150 and the patient. In some embodiments, the pump 180 may also pump dialysate from the dialysate bag 122 through, for example, a heating chamber. The controller 155 may also be operatively connected to a speaker 185 and a microphone 187 disposed in the machine 150. A user input interface 190 may include a combination of hardware and software components that allow the controller 155 to communicate with an external entity, such as a patient or other user. These components may be configured to receive information from actions such as physical movement or gestures and verbal intonation. In some embodiments, the components of the user input interface 190 may provide information to external entities. Examples of the components that may be employed within the user input interface 190 include keypads, buttons, microphones, touch screens, gesture recognition devices, display screens, and speakers. The machine 150 may also be wirelessly connectable via an antenna 192 for remote communication. The machine 150 may also include a display 195 and a power source 197.

As shown in FIG. 1B, the sensors 160 may be included for monitoring parameters and may be operatively connected, via a wired or wireless connection, to at least the controller 155, the processor 170, and/or the memory 175, or combinations thereof. The processor 170 may be configured to execute an operating system, which may provide platform services to application software (e.g., for operating the dialysis machine 150). These platform services may include inter-process and network communication, file system management and standard database manipulation. One or more of many operating systems may be used, and examples are not limited to any particular operating system or operating system characteristic. In some examples, the processor 170 may be configured to execute a real-time operating system such as RTLinux, or a non-real time operating system, such as BSD or GNU/Linux.

According to a variety of examples, the processor 170 may be a commercially available processor such as a processor manufactured by INTEL, AMD, MOTOROLA, and FREESCALE. However, the processor 170 may be any type of processor, multiprocessor or controller, whether commercially available or specially manufactured. For instance, according to one example, the processor 170 may include an MPC823 microprocessor manufactured by MOTOROLA.

The memory 175 may include a computer readable and writeable non-volatile data storage medium configured to store non-transitory instructions and data. In addition, the memory 175 may include a processor memory that stores data during operation of the processor 170. In some examples, the processor memory includes a relatively high performance, volatile, random access memory such as dynamic random-access memory (DRAM), static memory (SRAM), or synchronous DRAM. However, the processor memory may include any device for storing data, such as a non-volatile memory, with sufficient throughput and storage capacity to support the functions described herein. Further, examples are not limited to a particular memory, memory system, or data storage system.

The instructions stored in memory 175 may include data, executable programs or other code that may be executed by the processor 170. The instructions may be persistently stored as encoded signals, and the instructions may cause the processor 170 to perform the functions described herein. The memory 175 may include information that is recorded, on or in, the medium, and this information may be processed by the processor 170 during execution of instructions. The memory 175 may also include, for example, specification of data records for user timing requirements, timing for treatment and/or operations, historic sensor information, and the like. The medium may, for example, be optical disk, magnetic disk or flash memory, among others, and may be permanently affixed to, or removable from, the controller 155.

The sensor(s) 160 may include a pressure sensor for monitoring fluid pressure of the machine 150, although the sensors 160 may also include any of a heart rate sensor, a respiration sensor, a temperature sensor, a weight sensor, an air sensor, a video sensor, a thermal imaging sensor, an electroencephalogram sensor, a motion sensor, an audio sensor, an accelerometer, a capacitance sensor, or any other suitable sensor. It is appreciated that the sensors 160 may include sensors with varying sampling rates, including wireless sensors.

The controller 155 may be disposed in the machine 150 or may be coupled to the machine 150 via a communication port or wireless communication links, shown schematically as communication element 158. According to various examples, the communication element 158 may support a variety of one or more standards and protocols, examples of which include USB, Wi-Fi, TCP/IP, Ethernet, Bluetooth, Zigbee, CAN-bus, IP, IPV6, UDP, UTN, HTTP, HTTPS, FTP, SNMP, CDMA, NMEA and/or GSM. As a component disposed within the machine 150, the controller 155 may be operatively connected to any of the sensors 160, the pump 180, and the like. The controller 155 may communicate control signals or triggering voltages to the components of the machine 150. As discussed, exemplary embodiments of the controller 155 may include wireless communication interfaces. The controller 155 may detect remote devices to determine if any remote sensors are available to augment any sensor data being used to evaluate the patient.

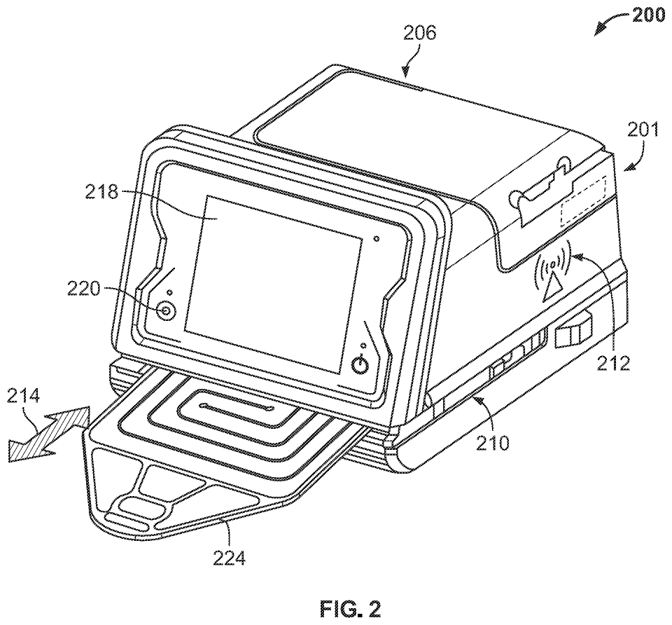

FIG. 2 illustrates an example of an embodiment of a dialysis machine 200 such as, for example, dialysis machine 150, that can be used in connection with the dialysis system 100 shown in FIG. 1A. The dialysis machine 200 may be implemented in the dialysis system 100 and may include, for example, a housing 206, a processing module 201, a connection component 212, a touch screen 218, and a control panel 220 operable by a user (e.g., a caregiver or a patient) to allow, for example, set up, initiation, and/or termination of a dialysis treatment. Some examples of commercially available dialysis machines 200 with which the disclosed embodiments could be used is the Versi™PD or the Liberty ® Select PD cyclers, manufactured and sold by Fresenius Medical Care of Waltham, Massachusetts.

The touch screen 218 and the control panel 220 may allow a user to input various treatment parameters to the dialysis machine 200 and to otherwise control the dialysis machine 200. In addition, the touch screen 218 may serve as a display. The touch screen 218 may function to provide information to the patient and the operator of the dialysis system 100. For example, the touch screen 218 may display information related to a dialysis treatment to be applied to the patient, including information related to a prescription.

The dialysis machine 200 may include a processing module 201 that resides inside the dialysis machine 200, the processing module 201 being configured to communicate with the touch screen 218 and the control panel 220. The processing module 201 may be configured to receive data from the touch screen 218, the control panel 220, and sensors (e.g., air, temperature and pressure sensors), and control the dialysis machine 200 based on the received data. For example, the processing module 201 may adjust the operating parameters of the dialysis machine 200. In some embodiments, the processing module 201 may be an MPC823 PowerPC device manufactured by Motorola, Inc.

Multiple Implementations of dialysate heating techniques include “batch” heating and/or “in-line” heating. For example, in an in-line heating embodiment (as illustrated), a warmer pouch 224 may be insertable into an internal chamber 210 in a direction indicated at arrow 214. It is also understood that the warmer pouch 224 may be connectable to the dialysis machine 200 via tubing, or fluid lines, via a cassette or cartridge. The tubing may be connectable so that dialysate may flow from the dialysate bags 122, through the warmer pouch 224 for heating, and to the patient.

In such in-line heating embodiments, the warmer pouch 224 may be configured so dialysate may continually flow through the warmer pouch (instead of transferred in batches for batch heating) to achieve a predetermined temperature before flowing into the patient. Internal heating elements (not shown) may be positioned so that when the warmer pouch 224 is inserted into the chamber 210, the one or more heating elements may affect the temperature of dialysate flowing through the warmer pouch 224. In some embodiments, the internal warmer pouch may instead be a portion of tubing in the system that is passed by, around, or otherwise configured with respect to, a heating element(s). It is understood that, in various implementations, dialysate may be transferable to and stored in one or more of the bags 122 (e.g., a heater bag) by “batch” until reaching an acceptable temperature for use and/or that dialysate continuously flowing through the warmer pouch 224 “in-line” with the dialysis machine 200, reaching an acceptable temperature by the application of internal heating elements.

The dialysis machine 200 may be configured to connect to a network. The connection to network may be via a wired and/or wireless connection. The dialysis machine 200 may include a connection component 212 configured to facilitate the connection to the network. The connection component 212 may be a transceiver for wireless connections and/or other signal processor for processing signals transmitted and received over a wired connection. Other medical devices (e.g., other dialysis machines) or components may be configured to connect to the network and communicate with the dialysis machine 200.

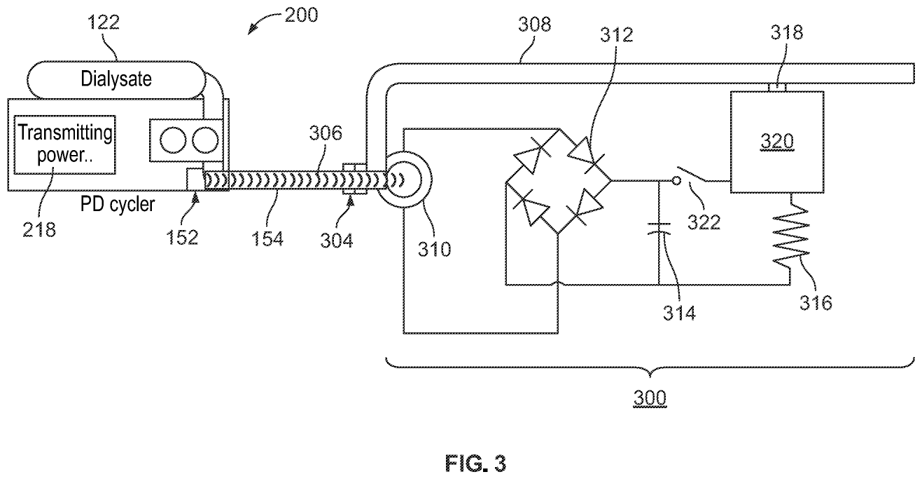

FIG. 3 is a schematic diagram of an exemplary embodiment of the present disclosure suitable with use with any system that transfers fluid as part of its normal operation, such as the PD dialysis machines 150, 200 shown in FIGS. 1A, 1B and 2. Various exemplary embodiments of the present disclosure are capable of powering or charging a remote device, for example, a remote sensor, using ultrasonic waves transmitted through a fluid carried in a conduit between a main unit and the remote device. As previously noted, although the invention is being presented in the context of a dialysis machine 200 and a remote pressure sensor, the invention may be used to power or charge any remote device fluidly coupled to another unit.

Dialysis machine 200 is shown in FIG. 3 with a reservoir of dialysate 122 which is pumped to a patient via cycler tubing 154. Cycler tubing 154 connects to catheter tubing 308 via tubing connection 304 as part of the normal PD therapy. Cycler tubing 154 may be, for example, disposable rubber tubing. Dialysis machine 200 is configured with an ultrasonic head 152 capable of generating ultrasonic waves 306. Device 300 is configured with a piezoelectric transducer 310, preferably disposed near tubing connection 304. Ultrasonic waves 306 generated by ultrasonic head 152 are transmitted by the fluid in cycler tubing 154 to device 300 and impinge on a surface of piezoelectric transducer 310. In certain embodiments, piezoelectric transducer 310 may be a commercially-available, off-the-shelf component. The frequency of ultrasonic waves generated by ultrasonic head 152 may be dependent upon the characteristics of piezoelectric transducer 310 and the characteristics of the particular fluid being pumped through cycler tubing 154. The described embodiments are not intended to be limited to a particular frequency of ultrasonic waves, nor to a particular fluid.

Device 300 may be any type of device and the second device to which it is connected and which generates the ultrasonic waves may also be any type of device fluidly connected to the device 300. In the context of the exemplary embodiment described herein, device 300 is a pressure sensor for sensing pressure in catheter tubing 308, the fluid is a dialysate and the second device is a dialysis machine 200. Device 300 is connected to dialysis machine 200 via cycler tubing 154. Operative components of device 300, in this embodiment, include a pressure sensor 318 for measuring pressure within catheter tubing 308 and a communication module 320 for communicating readings produced by pressure sensor 318 off-unit, for example, to dialysis machine 200 or to any other destination reachable via communication module 320. Pressure sensor 318 may be a commercially-available, off-the-shelf component selected specifically for measuring pressures in the expected range of pressures in catheter tubing 308. Communication module 320 is preferably a wireless module which may communicate by any known wireless protocol, for example, Wi-Fi, Bluetooth, NFC, etc. In some embodiments, device 300 may also be provided with a processor (not shown) executing software or firmware to carry out the intended purpose of device 300.

The kinetic energy of the ultrasonic waves conducted from dialysis machine 200 to device 300 via the fluid in cycler tubing 154 is converted to electrical power by the exemplary circuitry shown in FIG. 3. In this example, a full wave rectifier 312 converts AC power generated by piezoelectric transducer 310 into DC power which may be used to charge a power storage element, for example, capacitor 314, in device 300, or to directly power device 300. Preferably, the diodes used in rectifier 312 are composed of germanium to minimize voltage loss. In preferred embodiments, and in particular, when device 300 is implanted in the patient, the power storage element preferably comprises one or more capacitors 314. Alternatively, the power storage element may be a rechargeable battery (not shown) but, in particular for implantable medical devices, capacitors typically have a life span of 10-20 years versus 5-7 years for the rechargeable battery. Therefore, one or more capacitors 314 may be preferred as the power storage element(s). When device 300 is the type of device intended to be used with an APD cycler 200, capacitor 314 should be selected such as to be capable of completely charging in approximately 15-20 minutes, from the circuit's piezoelectric transducer 310. This is the typical fill phase time for an APD cycler 200, during which time the device 300 will be connected to dialysis machine 200 via cycler tubing 154. The circuitry may also include a current limiting resistor 316 to limit the current delivered to the communication module 320 and/or pressure sensor 318. It should be noted that the exemplary circuit shown in FIG. 3 is only one possible embodiment of circuitry for converting the kinetic energy of the ultrasonic wave to electrical power. Any other circuit implementation may be utilized. When device 300 stops receiving the ultrasonic energy (i.e., indicating that the patient has disconnected from the APD cycler and is in the dwell phase), switch 322 can be closed (automatically or manually) to begin discharging the capacitor to power pressure sensor 318 to monitor the pressure

In some embodiments, device 300 may be configured to only transmit data back to dialysis machine 200 when the tubing connection is reestablished and device 300 begins to recharge. In other embodiments, device 300 may transfer data in real time, during the dwell phase and when communication module 320 is powered by the power storage element. Readings from pressure sensor 318 may be wirelessly communicated by back to the APD cycler 200 or to a cloud-based server (not shown). Device 300 may include a series of capacitors to power different phases of data collection and subsequent data transmission. The APD cycler 200 or a cloud computing connection (not shown) would then use this data to facilitate building a model that optimizes dwell time.

Device 300 could be a wearable device on an external portion of catheter tubing 308 or it could be an implantable device.

Additional features for device 300 may include sensors for pressure, flow, temperature and even phase changes within these readings. This could be performed using ultrasonic sensors that measure through the adjacent tubing through reflection. This method could also be used to power blood pressure measurement, glucose, and activity sensors on or in the patient's body. Additionally, device 300 could interface with external sensors like a urinalysis device to identify the patient when in proximity to the delivered sample.

As would be realized by one of skill in the art, this technology could also be adapted for use in a non-APD cycler or as a clip-on module for a manual continuous ambulatory peritoneal dialysis (CAPD) system of bags and tubing. Additionally. It could be used to power an access site device for a hemodialysis patient where electrical isolation is also critical.

Various aspects of this described embodiments described herein have been explained in connection their use with dialysis machine 200 having a particular configuration. It is contemplated that the various aspects described herein may be used with dialysis machines having other configurations, for example, different types of dialysis machines. Further, it is contemplated that the described embodiments may include any type of device in fluid communication with a second device capable of generating the required ultrasonic waves and transmitting them via a fluid connection with device 300. The disclosed embodiments are intended to include both a device and a method to be performed by the device.

Some embodiments of the disclosed system may be implemented, for example, using a storage medium, a computer-readable medium or an article of manufacture which may store an instruction or a set of instructions that, if executed by a machine (i.e., processor or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A device comprising:
a fluid connection to a second device; and
circuitry for converting kinetic energy of ultrasonic waves transmitted to the device through the fluid connection into electrical energy.

2. The device of claim 1, further comprising:
a power storage element to store the electrical energy.

3. The device of claim 1, further comprising:
one or more sensing elements; and
a communications module;
wherein the one or more sensors and the communications modules are powered by the electrical energy.

4. The device of claim 3, wherein the communication module broadcasts readings from the one or more sensors to the second device or to a cloud-based server.

5. The device of claim 1 wherein the circuitry comprises:
a piezoelectric transducer;
a rectifier; and
one or more capacitors to store the electrical energy.

6. The device of claim 1, wherein the second device generates the ultrasonic waves and transmits them to the device through the fluid connection.

7. The device of claim 6, wherein the device is connected to the second device via tubing carrying the fluid connection.

8. The device of claim 7, wherein the second device is a peritoneal dialysis machine and further wherein the fluid connection comprises a dialysate.

9. The device of claim 8, wherein the one or more sensors comprises a pressure sensor for sensing pressure of the dialysate within a catheter of a patient.

10. The device of claim 9, wherein the device is implanted in the body of the patient.

11. A method for powering a device comprising:
receiving ultrasonic waves via a fluid connection to a second device; and
converting kinetic energy of the ultrasonic waves into electrical energy.

12. The method of claim 11, further comprising:
storing the electrical energy in a power storage element.

13. The method of claim 12, wherein the device comprises: further compromising:
one or more sensing elements; and
a communications module;
the method further comprising powering the one or more sensors and the communications modules using the stored electrical energy.

14. The method of claim 13, wherein the communication module broadcasts readings from the one or more sensors to the second device or to a cloud-based server.

15. The method of claim 11, wherein step of converting the kinetic energy of the ultrasonic waves into electrical energy is performed by circuitry comprising:
a piezoelectric transducer;
a rectifier; and
one or more capacitors to store the electrical energy.

16. The method of claim 11, wherein the second device generates the ultrasonic waves and transmits them to the device through the fluid connection.

17. The method of claim 16, wherein the device is connected to the second device via tubing carrying the fluid connection.

18. The method of claim 17, wherein the second device is a peritoneal dialysis machine and further wherein the fluid connection comprises a dialysate.

19. The method of claim 18, wherein the one or more sensors comprises a pressure sensor, the method further comprising:
sensing pressure of the dialysate within a catheter of a patient; and
broadcasting the sensed pressure via the communication module.

20. The method of claim 19, wherein the device is implanted in the body of the patient.
